(19) United States
(12) Patent Application Publication
(10) Pub. No.: US 2013/0244309 A1
(43) Pub. Date: Sep. 19, 2013

(54) Algal Floway (AGF) System for Economical and Efficient Harvesting of Algal Biomass and Method of Use

(71) Applicant: UNIVERSITY OF GEORGIA RESEARCH FOUNDATION, INC., Athens, GA (US)

(72) Inventors: Manjinder Singh, Athens, GA (US); Keshav C. Das, Athens, GA (US)

(73) Assignee: University of Georgia Research Foundation, Inc., Athens, GA (US)

(21) Appl. No.: 13/827,879

(22) Filed: Mar. 14, 2013

Related U.S. Application Data
(60) Provisional application No. 61/611,622, filed on Mar. 16, 2012.

(51) Int. Cl. C12M 1/00; C12N 1/12

(52) U.S. Cl. CPC C12M 29/04; C12N 1/12
USPC 435/257.1; 435/289.1; 435/297.2

(57) ABSTRACT

The present disclosure includes algal floway (AGF) systems for continuous, specific, economical, and efficient harvesting of algae biomass.

### CROSS-REFERENCE TO RELATED APPLICATION

This application claims priority to co-pending U.S. provisional application entitled “Algal Floway (AGF) System for Economical and Efficient Harvesting of Algal Biomass and Method of Use,” having Ser. No. 61/611,622 filed on Mar. 16, 2012, which is entirely incorporated herein by reference.

### STATEMENT REGARDING FEDERALLY SPONSORED RESEARCH OR DEVELOPMENT

This invention was made with Government support under grant numbers DEFG3608G08814 and DEFG3608G018164, awarded by the U.S. Department of Energy. The Government has certain rights in this invention.

### BACKGROUND

Algae are potential candidates for production of advanced biofuels in view of growing global energy concerns. Microalgae offer great promise to contribute a significant portion of the renewable fuels that will be required to meet the U.S. biofuel production target of 36 billion gallons by 2022, out of which 21 billion gallons should be from advanced biofuels as mandated in the Energy Independence and Security Act of 2007 under the Renewable Fuels Standard. In order to meet 100% of the mandated requirement of advanced biofuels, 4.3 million ha of algal ponds are needed.

Algal harvesting is typically energy intensive due to the concentration steps and dewatering required for the end products. Different approaches to harvesting and dewatering algae exist; these include, for example, centrifugation, flocculation (induced and auto), filtration and screening, flotation, gravity settling, and electrocoagulation. However, the cost and complexity of currently available options negatively impacts the potential economic, environmental, and social sustainability bottom line of using algae as a solution to problems.

### SUMMARY

Embodiments of the present disclosure, in one aspect, relate to algal floway systems for harvesting of algal biomass and methods of using these systems.

Briefly described, embodiments of the present disclosure include an algal floway (AGF) system comprising an algal floway structure, where the algal floway structure comprises: a tub, where the tub comprises a first sink on a first end of the tub, a second sink on an opposite end of the tub, and a horizontal surface between the first sink and the second sink, where the first sink comprises at least one opening on a base of the first sink for feeding algae culture from an algal cultivation reactor to the algal floway and where the second sink comprises at least one opening on a base of the second sink for draining algae culture from the algal floway to the algal cultivation reactor; a substrate on the horizontal surface of the tub; and at least one membrane on a surface of the substrate, where the at least one membrane supports the attachment and entrapment of algae; and a pump, where the pump circulates algae culture from the algae cultivation reactor through the AGF floway structure.

Embodiments of the present disclosure further include a method of harvesting algae biomass comprising circulating culture water from an algae cultivation reactor through an algal floway (AGF) system comprising a substrate and at least one membrane, where localized flow turbulence causes algae in the culture water to attach to the surface of the at least one membrane, draining the culture water back into the algae cultivation reactor, and harvesting the algal biomass from the at least one membrane.

Embodiments of the present disclosure also include a method of using an algal floway (AGF) system selected from cleaning eutrophic water bodies, optimizing water usage in algal cultivation, reducing atmospheric CO₂, enabling access to clean water, enabling access to algal food sources for humans and other animals, improving the economy of algae based biofuels, and a combination thereof.

### BRIEF DESCRIPTION OF THE DRAWINGS

Many aspects of the disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

The patent or application file contains at least one drawing executed in color. Copies of this patent or patent application publication with color drawing(s) will be provided by the Office upon request and payment of the necessary fee.

FIG. 1 shows the overview of an embodiment of a bench-scale AGF installed in a greenhouse and connected to a raceway pond.

FIG. 2 is a schematic diagram of an AGF system connected to an algal pond. 1; algae pond, 2; direction of algae culture movement, 3; paddle wheel, 4; pump to feed AGF, 5; AGF mat for biomass harvesting, 6; sinks in tub for feed and drain.

FIG. 3 is a graph that illustrates biomass growth in a raceway pond during the AGF harvesting period.

FIG. 4 is a photograph of a scaled up AGF system in operation.

FIGS. 5A-5F are photographs that illustrate different designs of AGF devices used with or without biomass being harvested; FIGS. 5A-5B, metal screen; FIGS. 5C-5D, plastic screen; and FIGS. 5E-5F, ruffled steel screen.

FIG. 6 is a graph that illustrates the effect of the area of AGF on harvesting capability. One, two or three units of AGF were combined in series to extend the AGF area. Each unit equals about 0.29 m².

FIGS. 7A-7C are graphs that illustrate biomass harvesting profiles of AGF1 (A), AGF2 (B), and AGF3 (C) when operated continuously over 9 days period.

FIG. 8 is a graph that illustrates comparative analysis of average biomass harvesting capabilities of AGF1 (smooth metal screen), AGF2 (smooth plastic screen), and AGF3 (ruffled metal screen).

FIGS. 9A-9F are microscopic images of algae raceway culture after AGF harvesting (A, B & C) and biomass collected from AGF surface (D, E & F).

### DETAILED DESCRIPTION

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

### DEFINITIONS

As used herein, a raceway includes a closed loop recirculation channel that is about 0.3 m deep. In the raceway, mixing and circulation are produced by a paddlewheel or other device that can accomplish the same result.

As used herein, day time includes a time period between about 9:00 a.m. and 5:00 p.m. and night time includes 5:00 p.m. to 9:00 a.m. in the context of AGF harvesting of the present disclosure.

### Discussion:

In accordance with the purpose(s) of the present disclosure, as embodied and broadly described herein, embodiments of the present disclosure, in one aspect, relate to algal floway (AGF) systems for economical and efficient harvesting of algae biomass.

A key high cost bottleneck to algal commercialization is efficient harvesting. For example, there are significant challenges to producing algal biomass for production of biofuels. Typically, algae in raceways have a dry biomass cell density of about 1-2 g/L, and a large quantity of water has to be processed to remove a few kg of algae biomass.

Embodiments of the present disclosure include an algal floway based continuous harvesting system where culture liquid from the raceway (or equivalent algal growth system) is continuously re-circulated through the floway. In an embodiment, larger cells that are in the culture attach to the floway and are effectively screened from the primary culture. Once attached to the screen in the AGF, the biomass is removed mechanically away from the floway, thus clearing space for additional organisms to attach.

Embodiments of the present disclosure include a continuous and size selective AGF harvester that is a low energy input system that continuously separates larger algal cells from the culture medium without affecting the viability of other cells or the growth rate in the culture medium. Embodiments of the present disclosure are based on the principles of tangential flow filtration (TFF, bulk flow travels tangentially across the membrane while the cells remain in suspension) combined with exploiting the ability (or tendency) of some algal species to attach to surfaces under conditions of localized turbulence in flow. In an embodiment of the AGF system of the present disclosure, the pressure drop across the membrane is negligible and flow occurs only through gravitational force (which is a modification from conventional TFF). In another embodiment of the present disclosure, filter (e.g., membrane) clogging is not a constraint, and the cells collected are not damaged and remain viable for re-culturing.

In TFF, the feed is passed through membranes where the bulk flow occurs tangentially across the filter, with a fraction (permeate containing smaller particle size solids that are of interest) passing through the filter (orthogonally). Because the bulk flow occurs tangentially across the filter, it removes larger particles that bind the filter, thus continuously keeping the filter clean allowing for long duration operations. This bulk flow, which contains larger particles, is returned to the feed (as retentate).

In the AGF system of the present disclosure, the TFF concept is modified such that the membrane layer is either single or multiple layers and allows flow only in the tangential direction (i.e., no flow orthogonal to the membrane). The coarse nature of the membrane allows filamentous, flagellate, or large cells to be captured in its matrix, while smaller cells are returned to the raceway. In an embodiment of the present disclosure, the AGF system is enhanced when operated in the presence of organisms that have a greater tendency to attach to surfaces (e.g., Periphytons such as Scendescmus, Oscillatoria, Phormidium species).

In an embodiment of the present disclosure, the AGF system comprises an algal floway structure (e.g., a flat structure, a flat slightly inclined structure) comprising at least one membrane on its surface. Culture water from an algae cultivation reactor (e.g., raceway) is pumped to the surface of the AGF system, where it flows over/through it and returns to the raceway. While the water flows through the AGF system, localized flow turbulence causes certain algae to attach to the surface of the at least one membrane; a process initiated by adhesive polysaccharides generated by the algae. This process further enhances the capture of other algal cells on the surface of the membrane. In an embodiment, algal biomass is periodically removed from the at least one membrane surface through external means (e.g., scraping).

In an embodiment of the present disclosure, the algal cells that are removed by the AGF system (by the membrane) are larger cells (e.g., cells that are at a later stage of growth). This selective harvesting by the AGF system of the present disclosure preferentially removes cells beyond the log phase of growth, thus maintaining the growth rate in the raceway at a maximum.

Embodiments of the present disclosure are thus useful for continuous size-selective harvesting of algae from a variety of different algal cultivation reactors, applications in cleanup of eutrophic water bodies, optimizing water usage in algal cultivation, reducing atmospheric CO₂, enabling better access to clean water, enabling better access to algal food sources for humans and other animals, improving the economy of algae based biofuels, and providing opportunities for small businesses in developing areas.

Embodiments of the present disclosure include an AGF system that removes only those cells that are ready for harvesting and returns the culture water back to the raceways, thus, increasing economic and environmental sustainability.

Embodiments of the present disclosure include an algal floway (AGF) system comprising an algal floway structure, where the algal floway structure comprises a tub, where the tub comprises a first sink on a first end of the tub, a second sink on an opposite end of the tub, and a horizontal surface between the first sink and the second sink, where the first sink comprises at least one opening on a base of the first sink for feeding algae culture from an algal cultivation reactor to the algal floway and where the second sink comprises at least one opening on a base of the second sink for draining algae culture from the algal floway to the algal cultivation reactor; a substrate on the horizontal surface of the tub; and at least one membrane on a surface of the substrate, where the at least one membrane supports the attachment and entrapment of algae; and a pump, where the pump circulates algae culture from the algae cultivation reactor through the AGF floway structure. In an embodiment, the AGF system further comprises an algal cultivation reactor, where the algal cultivation reactor comprises a raceway.

In an embodiment of the present disclosure, the at least on membrane comprises a first membrane and a second membrane, where the first membrane comprises a geotextile fabric, and where the second membrane comprises at least one screen. In another embodiment, the substrate comprises an about 0.5 to 1.0 inch thick plastic sheet, and the substrate, the first membrane, and the second membrane comprise a three layered sandwich structure on the horizontal surface of the tub.

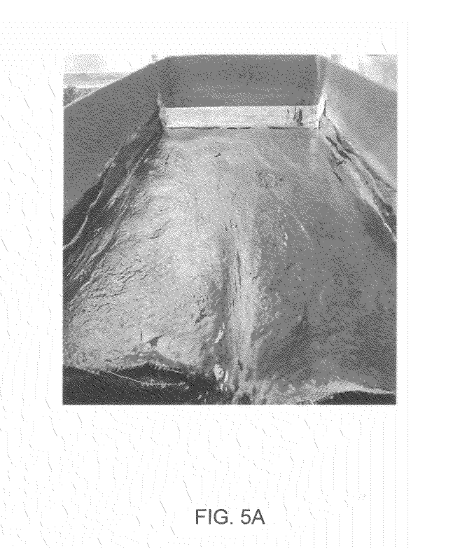

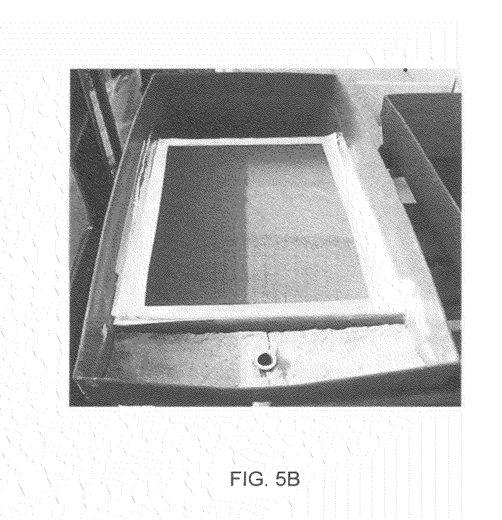

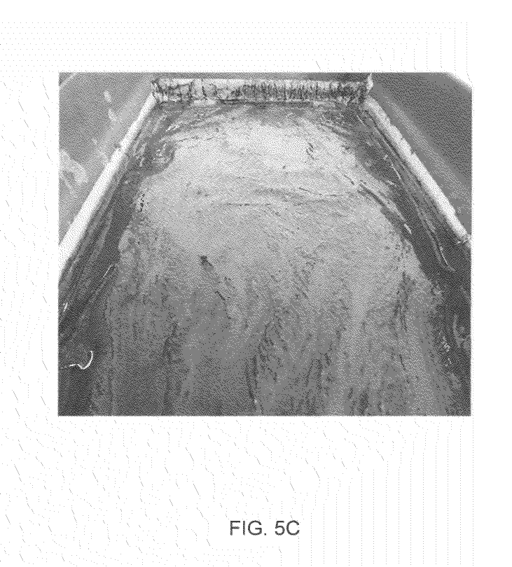

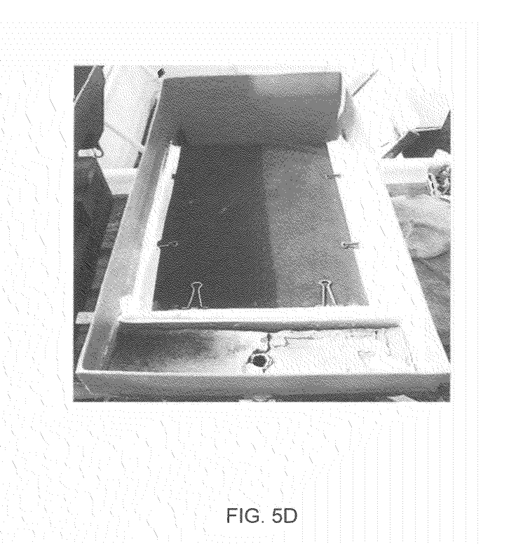

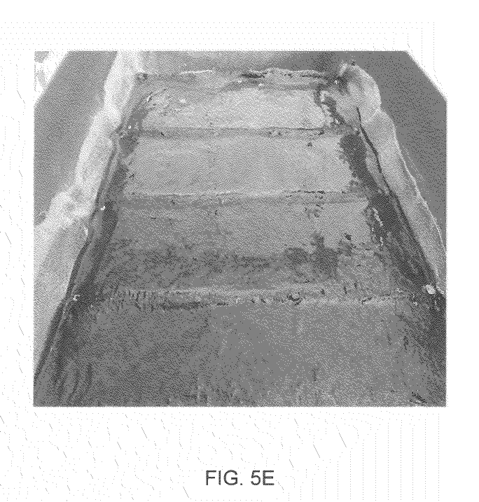

In an embodiment of the present disclosure, the plastic sheet is cut into dimensions such that it exactly fits into horizontal surface of tub. Then, the geotextile fabric and screen are cut into pieces which are about 1-2′ oversized on each side of the plastic sheet. Then, a three layered sandwich board is prepared with sequence of plastic-geotextile-screen, and the fabrics-screen layers are fixed onto the plastic sheet with steel screws and/or paper clips (FIG. 5D). This sandwich board is placed in the tub and the oversized fabric-screen edges are sealed against the sides of the tub by water resistant adhesive tape (FIG. 5B).

In an embodiment of the present disclosure, the algal floway structure is inclined at an angle of 0.1 to about 15 degrees.

Embodiments of the present disclosure include a screen is selected from a metal screen, a plastic screen, a metal riffled screen, a natural fabric, and a combination thereof. In an embodiment, the screen comprises a pore density of about 60-65 pores/sq. in. In another embodiment, the screen comprises a pore size of about 500 to 2000 microns. One skilled in the art would understand that the material for the substrate is selected for optimum attachment and entrapment of algal cells, especially in case of unicellular algae (e.g., a mesh material).

Embodiments of the present disclosure include a three layered sandwich structure where the surface area is about 5 to 15% of the surface area of the algae cultivation reactor.

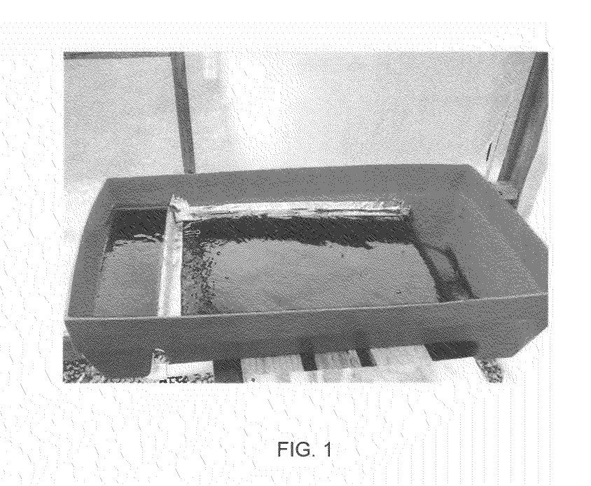

Embodiments of the present disclosure include a tub that is about 5 to 6 inches deep at its horizontal surface, and a first sink and a second sink that are each about 8 to 10 inches deep. In another embodiment, as shown in FIGS. 5B, 5D, and 5F, the first sink is filled with QUIKRETE® brand hydraulic water-stop cement slurry up to a level about 1″ below the overflow ridge of the first sink. This embodiment prevents settling and consequent degeneration of algae biomass in the first sink, which can happen when the sink is allowed to get filled before water starts overflowing the ridge (see FIG. 1).

In another embodiment of the present disclosure, the first sink has a depth of about 5 to 6 inches. One skilled in the art would understand that the depth of this first sink that precedes the floway is small and can be less than about 5 to 6 inches.

Embodiments of the present disclosure include a method of harvesting algae biomass comprising circulating culture water from an algae cultivation reactor through an algal floway (AGF) system comprising a substrate and at least one membrane, where localized flow turbulence causes algae in the culture water to attach to the surface of the at least one membrane, draining the culture water back into the algae cultivation reactor, and harvesting the algal biomass from the at least one membrane. In an embodiment, a submersible pump circulates the culture water through the AGF.

Embodiments of the present disclosure include a method of harvesting algae biomass further comprising turning on the submersible pump, filling the first sink by feeding algae culture from the algae cultivation reactor to the at least one opening on the base of the first sink so that the liquid algae culture overflows onto the horizontal surface and into the second sink, and draining the algae culture from the second sink through the at least one opening in the base of the second sink back to the algal cultivation reactor. In an embodiment, the flow rate across the floway is about 5 to 25 L/m²-min.

Embodiments of the present disclosure include a method of harvesting algae biomass where large sized, flocculated, or filamentous algae in the algae culture selectively attach to the at least one membrane as the culture flows over the at least one membrane. In an embodiment, the algal biomass is manually removed from the at least one membrane at regular intervals of time. In another embodiment, the algal biomass is manually removed from the at least one membrane about every 2 to 17 hours.

Embodiments of the present disclosure include continuously harvesting algal biomass at a rate of at least about 7 times the growth rate of the algae in the algae cultivation reactor. In an embodiment, the algal biomass harvested comprises an algal slurry of about 3 to 15% solids. In another embodiment, the algal biomass is removed at a rate of about 60 to 400 g/m² (harvester area)/d.

Embodiments of the present disclosure include an algal floway system where the horizontal surface area is about 0.25 m² to about 2.50 m². In an embodiment, the horizontal surface area is about 0.26 m². One skilled in the art would understand that the specific size could be optimized based on pumping energy and evaporation, among other variables.

Embodiments of the present disclosure include a membrane comprising a screen. In an embodiment, the membrane comprises a plastic screen which includes a fiberglass charcoal screen material of 63 mesh size (squared pores sq in⁻¹) (e.g., normally used as window or patio screen). In another embodiment, the screen is a metal screen which includes a bright aluminum screen wire of mesh size 63.

### EXAMPLES

### Example 1

### Description of Advanced Algal Floway (AGF) Biomass Harvester

The AGF comprises a shallow plastic tub with relatively deep sinks on two opposite sides. FIG. 1 shows the overview of the bench-scale AGF installed in a greenhouse and connected to a raceway pond.

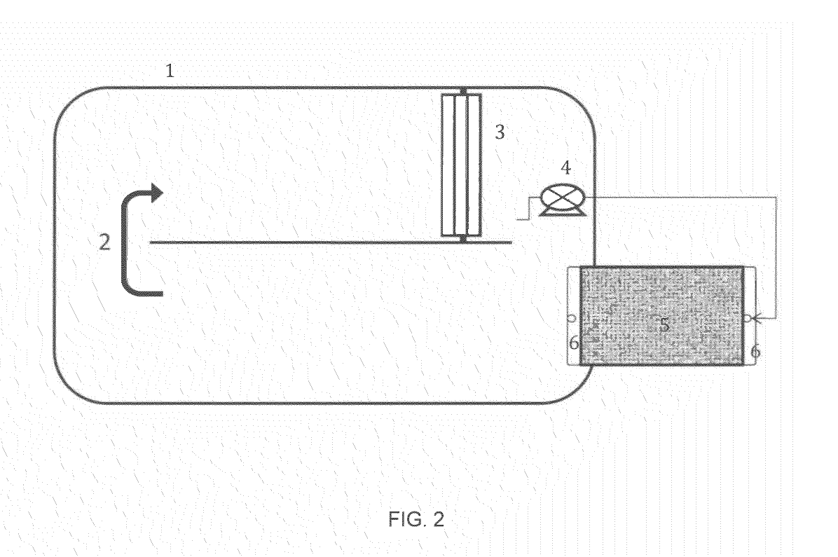

The schematic representation of the AGF system connected to an algae pond is shown in FIG. 2. The sinks have openings on their base for feeding and draining purposes. A solid material (substrate) is placed horizontally on the surface of the tub. The substrate supports the attachment and entrapment of algae. The horizontal surface areas of the tub and the material are the same which are about 0.26 m². A submersible pump is provided to circulate the algae culture over the AGF. As the Feed sink is filled from the base, the liquid overflows onto the AGF surface evenly when the pump in switched on.

The mechanics of the reactor system is that, medium which is circulated from the pond flows through the substrate and drains back to the pond. The algal cells that are larger in size (and potentially older in age) in the circulating culture liquid get entrapped and attached as a thin biofilm on the substrate which eventually turns into a thick biomat later. The algal biomat is harvested as thick slurry of about 3-8% solids by a mechanical scraping process.

### Algae Cultivation

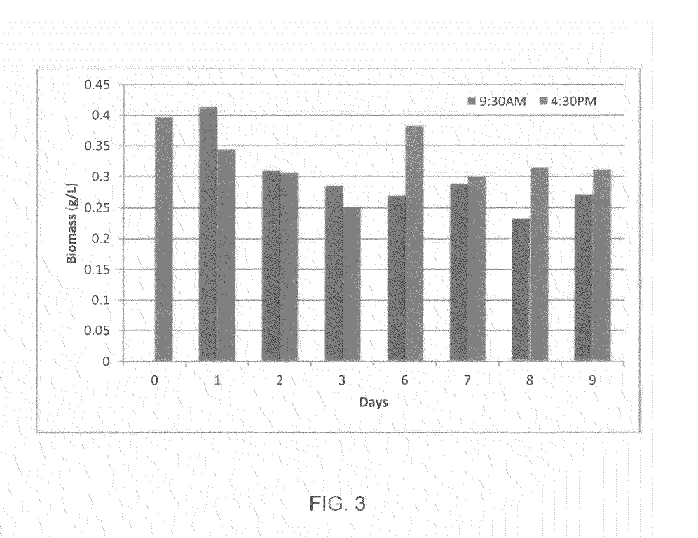

A mixed algae culture dominated by Chlorella sorokiniana, Chlorella minutissima and Scenedesmus bijuga was grown in raceway pond of surface area of about 2.9 m² (about 500 L culture volume) using BG11 standard growth medium. The biomass growth was measured gravimetrically as well as measuring optical density (OD) at 750 nm (FIG. 3). Once the biomass reached steady state concentration of about 0.3-0.4 g/L, the AGF system was connected and operated for about 10 days continuously.

### Biomass Harvesting

The continuous circulation of algae culture over the AGF resulted in development of a thick biomat of cells because of active attachment of algal cells as biofilm and passively as flocs. Duplicate AGF harvesters were operated to generate statistically significant results. The AGF was harvested twice during day time hours each day. To harvest, the pump was switched off and the water was allowed to drain for about 20-30 min so as to harvest thick biomass slurry from the AGF. The biomass was harvested manually using a plastic scrapper and transferred to a pre-weighed aluminium tray. The wet weight of the tray+biomass was measured. The biomass was then dried at about 80° C. until constant weight was observed after regular weighing. The dry weight and percentage solids in the wet biomass harvest were then calculated.

The results presented in Table 1 showed that about 70.8 g m⁻² of AGF d⁻¹ (about 6.5 g ft² of AGF d⁻¹) of dry weight biomass can be harvested by the AGF system when setup at a ratio of surface areas of AGF:Raceway 1:6. Considering the sustained algae biomass productivities of about 10 to 20 g m⁻² day⁻¹ in full scale open raceway ponds, the AGF foot prints are a fraction of the raceway footprints. Extending the harvesting over the night, where photosynthetic growth does not occur, can further double the harvesting efficiency and hence reduce the AGF foot prints.

TABLE 1
Biomass harvesting from steady state algae growth
in raceway pond by AGF system

| | Biomass harvested (dry g) | | |
|---|---|---|---|
| Days | AGF 1 | AGF 2 | Mean |
| 1 | 6.7 | 8 | 7.35 |
| 2 | 40.2 | 27.8 | 34 |
| 3 | 25 | 20.5 | 22.75 |
| 4 | 24.2 | 18.3 | 21.25 |
| 5 | 12 | 12.3 | 12.15 |
| 6 | 12 | 12.3 | 12.15 |
| 7 | 12 | 12.3 | 12.15 |
| 8 | 27.9 | 24.6 | 26.25 |
| 9 | 21.3 | 17 | 19.15 |
| 10 | 20.1 | 13.9 | 17 |
| Total (g) | | 368.4 | |
| Days | | 10 | |
| Per day (g) | | 36.84 | |

| | |
|---|---|
| AGF area (m²) | 0.52 |
| Harvest rate g/m²/d) | 70.8 |

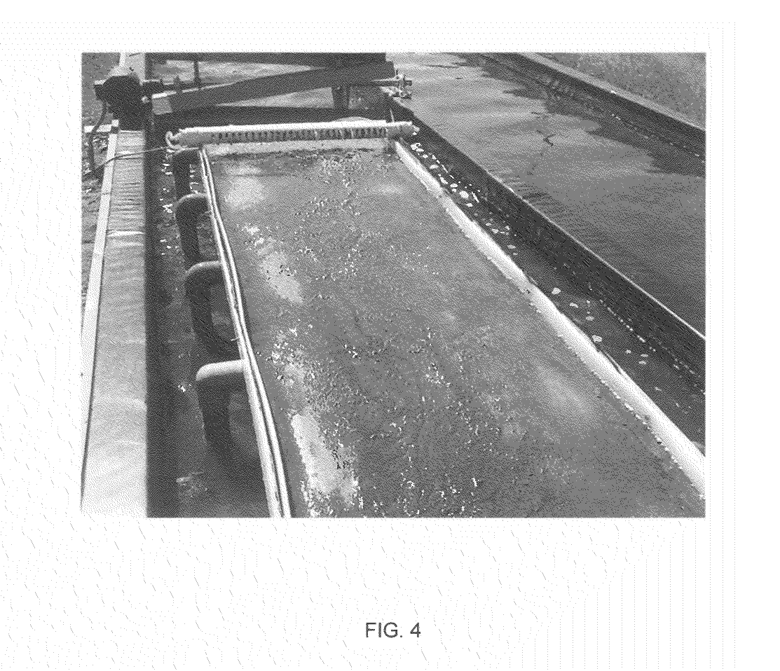

The AGF system was then scaled-up to about 2.23 m² harvesting area and tested with pilot scale raceway pond of about 100 m² (FIG. 4). Results obtained from data collected from 25 days operation which included 19 AGF harvests is presented in Table 2. The results were reproduced with a harvesting rate of about 73 g m⁻² harvest day⁻¹.

The AGF biomass harvesting technology of the present disclosure can significantly reduce costs of harvesting and eliminate down-stream dewatering costs. The about 8% solids biomass collected from AGF can directly be processed either via hydrothermal liquefaction or anaerobic digestion processes. The process can readily be scaled-up and automated (e.g., conveyor belt) for economical biomass harvesting.

TABLE 2
Biomass harvesting from steady state algae growth
in a raceway pond using the AGF system

| | Harvest |
|---|---|
| Day | (dry g) |
| 1 | 422 |
| 2 | 168 |
| 3 | 163 |
| 4 | 232 |
| 5 | 150 |
| 6 | 159 |
| 7 | 168 |
| 8 | 209 |
| 9 | 82 |
| 10 | 132 |
| 11 | 168 |
| 12 | 186 |
| 13 | 95 |
| 14 | 91 |
| 15 | 95 |
| 16 | 114 |
| 17 | 150 |
| 18 | 177 |
| 19 | 132 |
| Total (g) | 3093 |
| Times harvest | 19 |
| Per harvest | 162.8 |
| AGF Area (m²) | 2.23 |
| Harvest rate (g/m²/harvest) | 73.0 |
| Total Days of operation | 25 |
| Harvest/day | 123.72 |
| Harvest rate (g/m²/d) | 55.5 |

### Example 2

This study was carried out for four months to optimize different working parameters for enhancing algal biomass harvesting capability of the AGF device. A total of three AGF units each of about 0.29 m² harvesting area were used. Biomass harvesting capacity of individual units and in combinations of 2 or 3 AGF units, when connected in series for water overflow, was tested. AGFs were run in batches of about 7-10 days for evaluating any specific parameter. Biomass harvesting capacity in terms of percentage solids and dry algae biomass g m⁻² d⁻¹ were calculated from biomass slurry harvested twice a day by manual scraping method. The following parameters were analyzed and evaluated:
1. Area of AGF: about 0.29 m² (one unit), about 0.58 (two units) and about 0.87 m² (three units)
2. Type of harvesting screen: metal, plastic and metal ruffled (FIGS. 5A-5F).
3. Period of harvesting: about 7 hr (day time) and about 17 hr (night time).

During initial batch, all three AGFs were fitted with previously tested plastic screen and were operated in combination. They were designated as AGF1, AGF2 & AGF3 as per their position in the series. Algae culture from raceways growing algae in compost leachate pond water was pumped over to AGF1 followed by AGF 2 and AGF 3 before returning back to the original raceway pond. All experiments were performed under greenhouse canopy. AGFs were harvested two times in a day. Morning harvest represent biomass collected over AGFs surface during about 17 hrs operation at night while Evening harvest represent about 7 hr biomass collection during day time operation of AGFs.

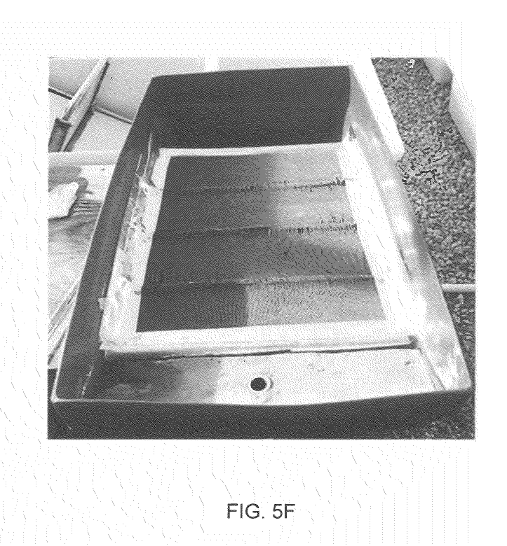

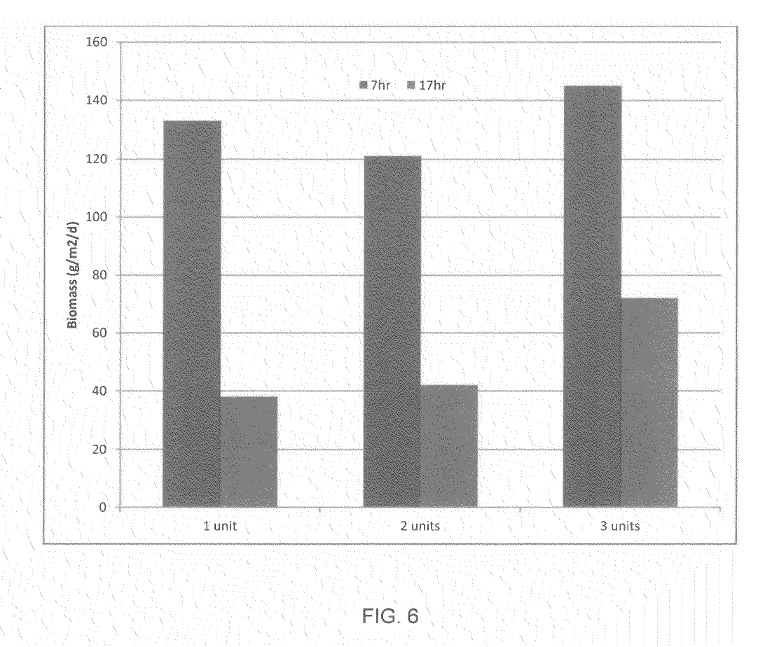

When operating 3 units in combination using plastic screen without surface modification, an average of about 145 and 72 dry grams of algae m⁻² d⁻¹ were harvested during about 7 and 17 hr operation respectively (FIG. 6). These and all other results achieved from different batches throughout this study revealed a common trend that about 7 hr harvesting is far better than about 17 hr harvesting period. Harvesting after about 7 hr biomass collection over AGF surface produced 2-3 times more biomass than when AGFs were allowed to run for about 17 hr prior to harvest. More frequent harvest produces more biomass. In a specific trial during this study the AGFs were harvested every about 3 hrs and biomass up to about 400 g m⁻² d⁻¹ was removed from a single AGF unit. Therefore, the optimized method for scale-up automated AGF system should consider frequent harvesting after regular short intervals (e.g., harvest after about 3-4 hrs). Increasing surface area increased the harvesting capacity of AGF, however, the biomass yield in terms of g/m⁻² d⁻¹ was not affected significantly when single or multiple units were operated in combinations.

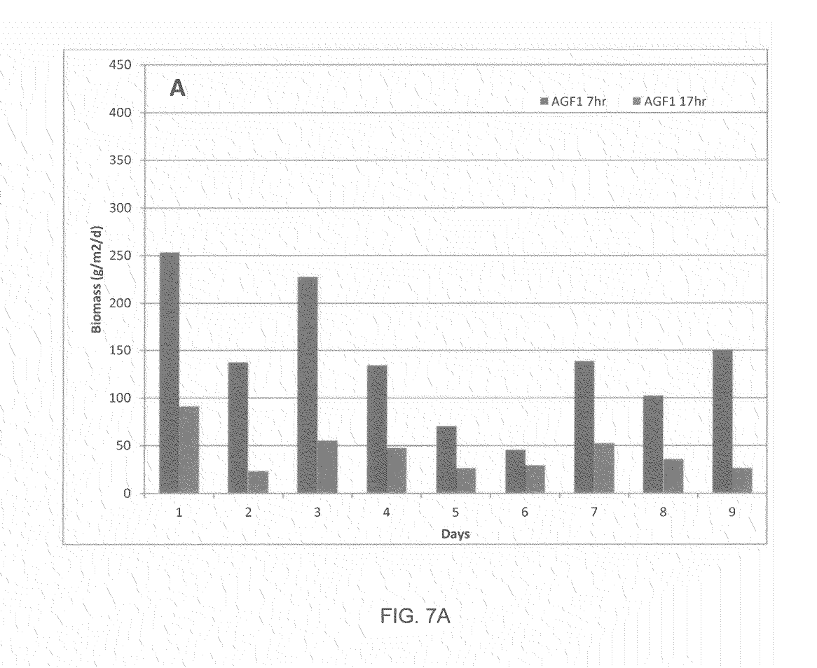

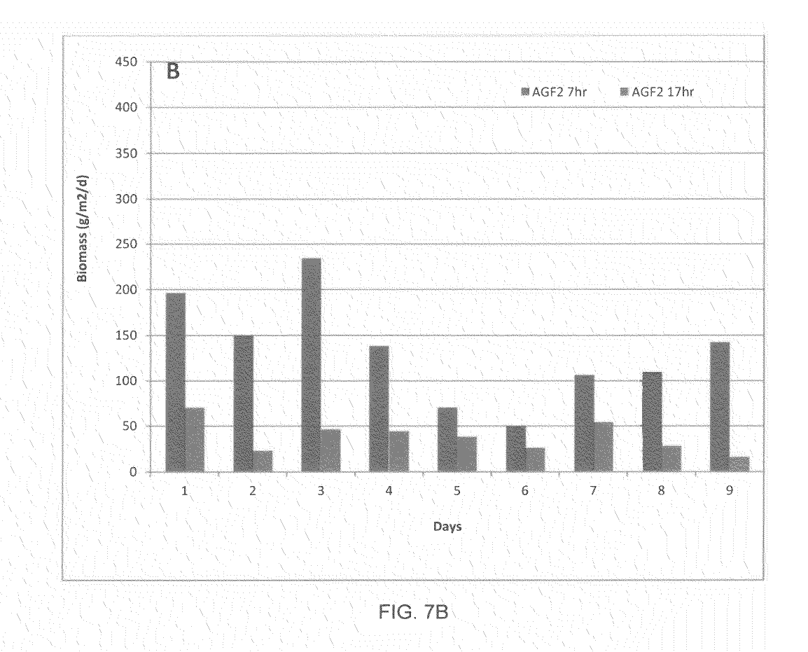

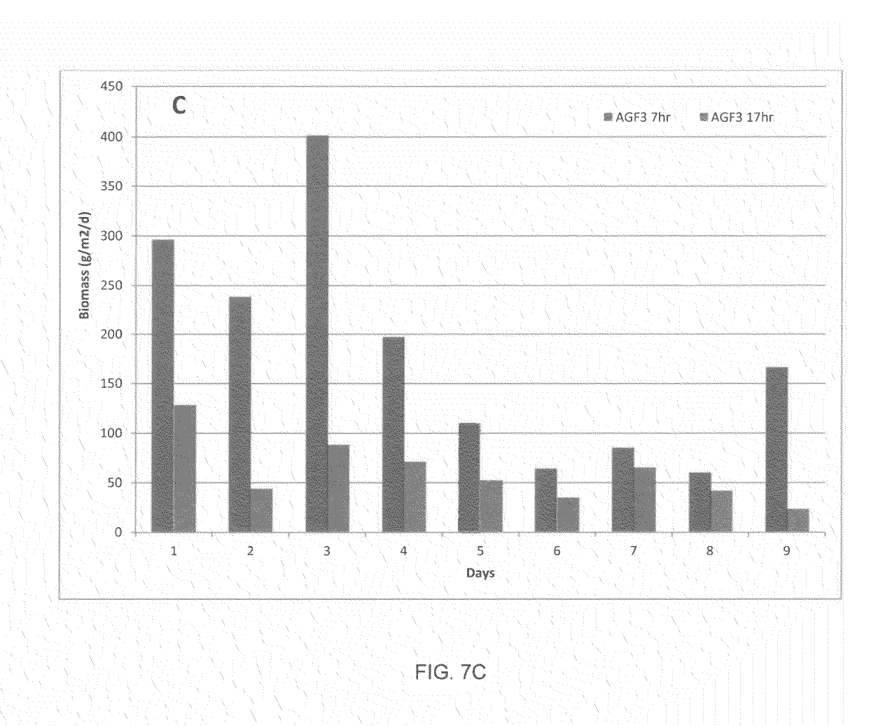

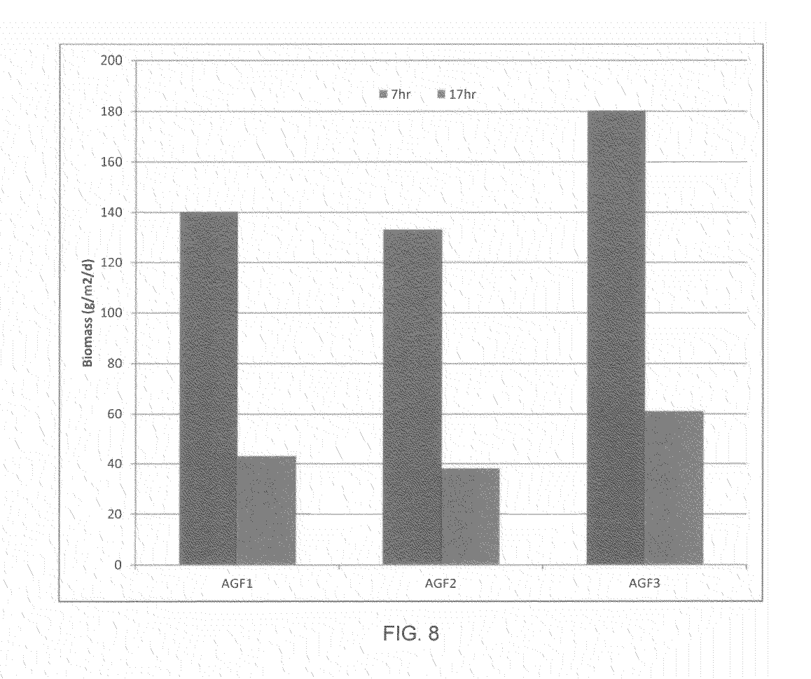

The main parameter optimized was using different types of harvesting screens. Three types of screen dimensions were tested. AGF1, 2 and 3 were refitted with metal smooth surface, plastic smooth surface and metal ruffled surface screens respectively (FIGS. 5A-5F). AGFs were harvested individually and biomass yield was calculated separately for three AGFs to evaluate the role of screen in harvesting. FIGS. 7 and 8 show the results achieved for this experiment. As was hypothesized, AGF 3 with ruffled steel screen produced significantly higher yield when compared to AGF 1 and 2. The results of AGFs 1 and 2 when compared for biomass harvesting capability were found to be statistically insignificant. A maximum of about 400 gm⁻² d⁻¹ was achieved by raffled metal screen AGF (3) during about 7 hr collection period.

It's worth noting here that biomass harvesting capacity of AGFs also depend on algae culture density and nature of algal culture. Higher cell density yield more biomass over AGF. Similarly, filamentous and flocculated algae are better harvested by the AGF system.

### Example 3

The first parameter optimized was the type of support material used for harvesting algae biomass. Two different types of support materials viz. plastic and metal mesh screens were tested. These types are shown in FIG. 5.

AGFs were operated continuously with algae raceways and analytical data was collected for 9 days batch period. Results achieved revealed that there was no statistically significant difference in the performance of each support material type in terms of biomass harvesting capability. However, metal screen was found to be more durable and stable as compared to plastic screen. Plastic screen tore out more frequently with continuous scraping activities. Therefore, metal screen was selected for further optimization studies.

The second optimization parameter was modification of surface area of the harvesting screen to enhance the biomass yield. Again two deigns: Smooth surface and raffled surface were developed and compared. It was hypothesized that an uneven surface of AGF will slow down water flow and will allow more biomass to settle down on the AGF surface, which will eventually result in enhanced biomass harvesting capability of the system. Our hypothesis was proved true as results obtained from 9 days parallel operation of both types of AGF surfaces showed that ruffled AGF achieved about 29% more biomass harvesting than smooth surface AGF.

The third parameter evaluated throughout this study was the biomass harvesting period. It was the period of time between two consecutive AGF harvestings. AGFs were operated continuously and were harvested twice a day at 9:30 am and 4:30 pm corresponding to about 17 h and about 7 h biomass accumulation time respectively. Our hypothesis was that the AGF surface has certain limits of collecting biomass and as it approaches that limit, the rate of biomass collection slows down resulting in lower biomass harvesting rate (g/d). A more frequent biomass removal from the surface of the AGF will re-charge the surface for more biomass collection. The about 7 h harvesting removed about 2-3 times more biomass (m⁻² d⁻¹) as compared to the about 17 h operation. An average of about 180 g m² d⁻¹ was achieved with raffled metal screen AGF harvested after about 7 h during continuous operation.

This system selectively harvests old, large sized, flocculated and filamentous algae from continuous algae growth system. As algal cells grow old they tend to increase in size, grow slowly and tend to flocculate as clumps. If not separated from an active algae growth system, they tend settle at the bottom of the pond getting degenerated, which may result in a pond crash or overall slow growth. Removing such fat and old algae serves two purposes: it clears the growth system from old cells and provides more chances to young cells to grow and produce more biomass. The AGF system is an ideal harvester for such algae, as it selectively removes flocculated and large sized algal cells.

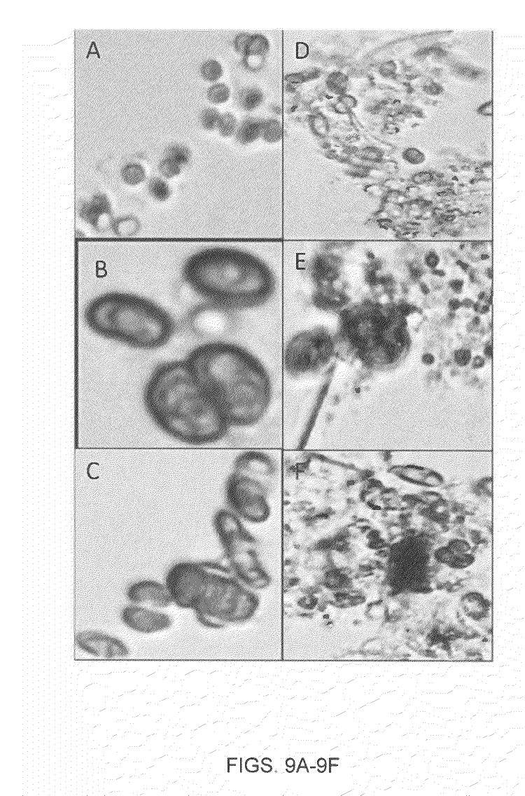

When the AGF was operated with highly flocculated algae culture, a maximum of about 401 gm⁻² d⁻¹ of biomass removal was achieved. To achieve this, the AGF was harvested about every 3 h to avoid saturation of AGF surface. To further prove this, AGF harvested biomass and pond culture after AGF harvest were analyzed using microscopic techniques. FIG. 9 shows the microscopic images captured from algae culture left in the raceways pond after AGF harvesting (images A to C) and those captured from biomass collected from the surface of AGF (images D to F). It is evident that AGF removed more flocculated, filamentous and fragmented algal cells, leaving behind individual and healthy cells in the pond.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include ±1%, ±2%, ±3%, ±4%, ±5%, ±6%, ±7%, ±8%, ±9%, or ±10%, or more of the numerical value(s) being modified. In an embodiment, the term “about” can include traditional rounding according to the numerical value. In addition, the phrase “about ‘x’ to ‘y’” includes “about ‘x’ to about ‘y’”.

It should be emphasized that the above-described embodiments of the present disclosure are merely possible examples of implementations, and are merely set forth for a clear understanding of the principles of the disclosure. Many variations and modifications may be made to the above-described embodiments. All such modifications and variations are intended to be included herein within the scope of this disclosure and protected by the following claims.

Therefore, at least the following is claimed:

1. An algal floway (AGF) system comprising:
an algal floway structure, wherein the algal floway structure comprises:
a tub, wherein the tub comprises a first sink on a first end of the tub, a second sink on an opposite end of the tub, and a horizontal surface between the first sink and the second sink, wherein the first sink comprises at least one opening on a base of the first sink for feeding algae culture from an algal cultivation reactor to the algal floway and wherein the second sink comprises at least one opening on a base of the second sink for draining algae culture from the algal floway to the algal cultivation reactor;
a substrate on the horizontal surface of the tub; and
at least one membrane on a surface of the substrate, wherein the at least one membrane supports the attachment and entrapment of algae; and
a pump, wherein the pump circulates algae culture from the algae cultivation reactor through the AGF floway structure.

2. The AGF system of claim 1, further comprising an algal cultivation reactor, wherein the algal cultivation reactor comprises a raceway.

3. The AGF system of claim 1, wherein the at least on membrane comprises a first membrane and a second membrane, wherein the first membrane comprises a geotextile fabric, and wherein the second membrane comprises at least one screen.

4. The AGF system of claim 3, wherein the substrate comprises an about 0.5 to 1.0 inch thick plastic sheet, and wherein the substrate, the first membrane, and the second membrane comprise a three layered sandwich structure on the horizontal surface of the tub.

5. The AGF system of claim 1, wherein the algal floway structure is inclined at an angle of 0.1 to about 15 degrees.

6. The AGF system of claim 3, wherein the screen is selected from the group consisting of: a metal screen, a plastic screen, a metal riffled screen, a natural fabric, and a combination thereof.

7. The AGF system of claim 6, wherein the screen comprises a pore density of about 60-65 pores/sq. in. and a pore size of about 500 to 2000 microns.

8. The AGF system of claim 4, wherein the surface area of the three layered sandwich structure is about 5 to 15% of the surface area of the algae cultivation reactor.

9. The AGF system of claim 1, wherein the tub is about 5 to 6 inches deep at its horizontal surface, and wherein the first sink and the second sink are each about 8 to 10 inches deep.

10. A method of harvesting algae biomass comprising:
circulating culture water from an algae cultivation reactor through an algal floway (AGF) system comprising a substrate and at least one membrane, wherein localized flow turbulence causes algae in the culture water to attach to the surface of the at least one membrane;
draining the culture water back into the algae cultivation reactor; and
harvesting the algal biomass from the at least one membrane.

11. The method of claim 10, wherein a submersible pump circulates the culture water through the AGF.

12. The method of claim 11, wherein the AGF system further comprises a tub, wherein the tub comprises a first sink on a first end of the tub, a second sink on an opposite end of the tub, and a horizontal surface between the first sink and the second sink, wherein the first sink comprises at least one opening on a base of the first sink, and wherein the second sink comprises at least one opening on a base of the second sink, and further comprising:
turning on the submersible pump;
filling by feeding algae culture from the algae cultivation reactor to the at least one opening on the base of the first sink so that the liquid algae culture overflows onto the horizontal surface and into the second sink; and
draining the algae culture from the second sink through the at least one opening in the base of the second sink back to the algal cultivation reactor.

13. The method of claim 10, wherein the flow rate across the floway is about 5 to 25 L/m²-min.

14. The method of claim 10, further comprising size selective harvesting, wherein large sized, flocculated, or filamentous algae in the algae culture selectively attach to the at least one membrane as the culture flows over the at least one membrane.

15. The method of claim 14, further comprising manually removing the algal biomass from the at least one membrane at regular intervals of time.

16. The method of claim 15, further comprising manually removing the algal biomass from the at least one membrane about every 2 to 17 hours.

17. The method of claim 10, further comprising continuously harvesting the algal biomass at a rate of at least about 7 times the growth rate of the algae in the algae cultivation reactor.

18. The method of claim 10, wherein the algal biomass comprises an algal slurry of about 3 to 15% solids.

19. The method of claim 10, wherein the algal biomass is removed at a rate of about 60 to 400 g/m² (harvester area)/d.

20. A method of using an algal floway (AGF) system selected from the group consisting of: cleaning eutrophic water bodies, optimizing water usage in algal cultivation, reducing atmospheric CO₂, enabling access to clean water, enabling access to algal food sources for humans and other animals, improving the economy of algae based biofuels, and a combination thereof.
